(19) United States
(12) Patent Application Publication
(10) Pub. No.: US 2014/0112878 A1
(43) Pub. Date: Apr. 24, 2014

(54) Tyrosinase Inhibitor

(71) Applicant: Kao Corporation, (US)

(72) Inventors: Tsuyoshi OBA, Utsunomiya-shi (JP); Mamiko Kikuchi, Utsunomiya-shi (JP); Akira Hachiya, Utsunomiya-shi (JP)

(21) Appl. No.: 13/869,128

(22) Filed: Apr. 24, 2013

(30) Foreign Application Priority Data
Apr. 24, 2012 (JP) 2012-098659

(51) Int. Cl. A61K 31/05; A61K 8/34; A61Q 19/02

(52) U.S. Cl. CPC A61K 31/05; A61Q 19/02; A61K 8/347
USPC 424/62; 568/781; 514/731

(57) ABSTRACT

Provided is a tyrosinase activity inhibitor, a melanin production inhibitor, a skin-whitening agent, and a cosmetic product, which have an excellent tyrosinase activity inhibitory effect with a high level of safety.

A tyrosinase inhibitor comprising isopropylmethylphenol as an active ingredient.

### FIELD OF THE INVENTION

The present invention relates to a tyrosinase inhibitor, a skin-whitening agent, and the like, which suppress melanogenesis.

### BACKGROUND OF THE INVENTION

Tyrosinase is known as an enzyme which is involved in the biosynthesis of melanin. During the melanogenesis process, tyrosinase catalyzes the hydroxylation reaction which converts tyrosine into L-DOPA (3,4-dihydroxy-L-phenylalanine) and the oxidation reaction which converts L-DOPA into dopaquinone. Thus, tyrosinase directly affects the biosynthesis of melanin from tyrosine within an organism (Int. J. Mol. Sci. 2009, 10, 2440-2475).

Melanin, produced through the above process, serves to prevent UV damage to somatic cells within an organism. On the other hand, excessive level of melanogenesis is known to lead to skin tanning and pigmentation, thereby causing spots and freckles. This seems problematic not only from aesthetic viewpoint but also from healthetic viewpoint.

Therefore, conventionally, controlling of the tyrosinase activity has been suggested in order to suppress an excessive level of melanogenesis, and substances for inhibiting tyrosinase activity have been searched. For example, vitamin C, arbutin, and kojic acid are known as substances which inhibit tyrosinase activity.

Meanwhile, isopropylmethylphenol (also known as p-thymol) has bactericidal and antibacterial activity. Since isopropylmethylphenol acts on resident skin bacteria which tend to grow in sebum, the compound has conventionally been used for cosmetics, drugs and the like, in a concentration of 0.05 to 0.10% by mass (500 to 1000 ppm), as a bactericidal agent.

Thymol and carvacrol, both regioisomers of isopropylmethylphenol, are reported to exhibit inhibitory activity on mushroom-derived tyrosinase, and to exhibit suppressive effect on melanin production in mouse melanoma B-16 cells (JP-A-9-249544). However, inhibitory activity on a human-derived tyrosinase has not been reported, as well as there has been absolutely no report which suggests that isopropylmethylphenol exhibits inhibitory activity against tyrosinase and suppressive effect on melanin production.

### SUMMARY OF THE INVENTION

The present invention relates to the following (1) to (10).

(1) A tyrosinase inhibitor comprising isopropylmethylphenol as an active ingredient.
(2) A melanin production inhibitor comprising isopropylmethylphenol as an active ingredient.
(3) A skin-whitening agent comprising isopropylmethylphenol as an active ingredient.
(4) A cosmetic product comprising isopropylmethylphenol in a concentration of 0.00001 w/v % or more and 0.04 w/v % or less.
(5) Use of isopropylmethylphenol for the production of a tyrosinase inhibitor.
(6) Use of isopropylmethylphenol for the production of a melanin production inhibitor.
(7) Use of isopropylmethylphenol for the production of a skin-whitening agent.
(8) A method for inhibiting tyrosinase comprising administering or feeding isopropylmethylphenol to a subject in need thereof.
(9) A method for suppressing melanogenesis comprising administering or feeding isopropylmethylphenol to a subject in need thereof.
(10) A method for skin-whitening comprising administering or feeding isopropylmethylphenol to a subject in need thereof.

### DETAILED DESCRIPTION OF THE INVENTION

The present invention relates to the provision of a tyrosinase activity inhibitor, a melanin production inhibitor, a skin-whitening agent, and a cosmetic product, which have an excellent tyrosinase activity inhibitory effect with a high level of safety.

As a result of investigations to solve the above problem, the present inventors have found that, surprisingly, isopropylmethylphenol as a bactericidal agent contained in, for example, cosmetic products exhibits an excellent inhibitory effect on a human-derived tyrosinase activity. Moreover, the present inventors have also found that isopropylmethylphenol shows the inhibitory effect on a human-derived tyrosinase activity at a lower concentration than the effective concentration of isopropylmethylphenol conventionally used as a bactericidal agent.

The tyrosinase inhibitor, the melanin production inhibitor, the skin-whitening agent and the cosmetic product of the present invention enable to suppress an exceeding level of melanin production on the skin, thereby preventing or improving the pigmentations, spots and freckles after tanning.

As used herein, “tyrosinase inhibition” means the inhibition of tyrosinase activity. Here, tyrosinase is an enzyme involved in a key reaction of melanogenesis of mammals, and in a key reaction of an enzymatic browning of fruits and fungi. In the present invention, the tyrosinase is preferably, but not particularly limited to, a mammal-derived tyrosinase, and more preferably human tyrosinase. The activities of the tyrosinase include, for example, conversion of tyrosine into dihydroxy phenylalanine (DOPA), and conversion of DOPA into dopaquinone.

As used herein, “skin-whitening (effect)” means suppressing the production of melanin pigment to return the color of the skin to the original color of the skin without any extra melanin, or to prevent and suppress the skin tanning or pigmentation such as spots and freckles.

Isopropylmethylphenol used in the present invention refers to any isopropylmethylphenol which can be used in, for example, cosmetic products, drugs, quasi-drugs, and is not particularly limited to a specific isopropylmethylphenol. The isopropylmethylphenol may be chemically synthesized according to the publicly known method (for example, DE 102007035515), and commercially available products may also be used.

As hereinafter described in Examples, isopropylmethylphenol inhibits tyrosinase activity (DOPA oxidase activity) on human tyrosinase (neonatal epidermal melanocyte extract) (Example 1). The tyrosinase activity inhibitory effect was also confirmed in the culture system using human neonatal epidermal melanocyte (Example 2). As mentioned above, tyrosinase is an enzyme involved in the biosynthesis of melanin (ibid., Int. J. Mol. Sci. 2009, 10, 2440-2475). Therefore, isopropylmethylphenol can be said to exhibit a skin-whitening effect, by inhibiting tyrosinase activity and suppressing melanin production.

The tyrosinase activity inhibitory effect can be observed at a concentration of isopropylmethylphenol lower than the predetermined concentration (500 to 1000 ppm) of isopropylmethylphenol used as a bactericidal agent, and the level of the inhibitory effect is higher than that of kojic acid publicly known as a tyrosinase activity inhibitor.

Thus, isopropylmethylphenol can be used for the purposes of tyrosinase inhibition, melanin production suppression, or skin-whitening, at a low dosage. The use may be for both human and non-human animal use, and the purpose of the use may be both therapeutic and non-therapeutic use.

Furthermore, isopropylmethylphenol can be used as a tyrosinase inhibitor, a melanin production inhibitor, and a skin-whitening agent (hereinafter referred to as “tyrosinase inhibitor and the like”), and can also be used to produce these inhibitors and agent. Isopropylmethylphenol may be used alone, or in combination with, for example, an acceptable pharmaceutical carrier as necessity in the tyrosinase inhibitor and the like.

The tyrosinase inhibitor and the like themselves may be cosmetic products, quasi-drugs or drugs, having each of tyrosinase inhibition, melanin production suppression, and skin-whitening effects. The tyrosinase inhibitor and the like may also be used as a material or a formulation, contained in, for example, the cosmetic product.

Furthermore, the tyrosinase inhibitor and the like may be used as cosmetic products or quasi-drugs which are prepared based on concepts of tyrosinase inhibition, melanin production suppression or skin-whitening, and which have indications of such concepts upon necessity.

The above-mentioned drugs (including quasi-drugs) may be administered in any administration routes. The administration route may be either parenteral administrations such as suppositories, inhalation drugs, transdermal systems, and external preparations (lotion, milk lotion, gel, cream, ointment and the like), or oral administrations such as pills, capsules, granule, powder, and syrup, among them, parenteral administrations are preferable and external administrations are more preferable.

In order to prepare such various dosage forms of medicinal preparations, isopropylmethylphenol may be used alone, or in combination with a pharmaceutically acceptable excipient, binder, extender, disintegrant, surfactant, lubricant, dispersant, buffering agent, preservative, flavoring substance, perfume, coating material, carrier, or diluent, as necessary.

The above-mentioned cosmetic products (including quasi-drugs) may be in forms of, for example, skin external preparations, detergents, makeup cosmetics, and may be provided in various dosage forms such as lotion, milk lotion, gel, cream, ointment, powder, and granule, according to the usage. In order to prepare such various dosage forms of cosmetic products, isopropylmethylphenol may be used alone, or combined with materials contained in quasi-drugs, skin cosmetics, and cleansing products, such as oily ingredients, moisturizing agents, powdery agents, coloring agents, emulsifying agents, solubilizing agents, detergents, ultraviolet absorbing agents, thickening agents, medicinal ingredients, perfume, resins, anti-bacterial and anti-mold agents, botanical extracts, and alcohol, as necessary. The medicinal ingredients may be other skin-whitening components such as hormonal agents, kojic acids, arbutin, placenta extract, chamomile extract, and rucinol.

The content of isopropylmethylphenol in the total amount of each of the drugs, quasi-drugs and cosmetic products mentioned above, is generally at a concentration of 0.00001 w/v % or more and 0.04 w/v % or less, and from the viewpoint of tyrosinase inhibitory effect and safety, 0.00001 w/v % or more, preferably 0.0001 w/v % and more, still more preferably 0.0005 w/v % or more, still more preferably 0.0007 w/v % or more, still more preferably 0.001 w/v % or more, still more preferably 0.002 w/v % or more, and 0.04 w/v % or less, 0.02 w/v % or less, 0.01 w/v % or less, 0.005 w/v % or less, 0.003 w/v % or less, or 0.002 w/v % or less. And, for example, the content is 0.00001 to 0.04 w/v %, 0.0001 to 0.04 w/v %, 0.0005 to 0.04 w/v %, 0.001 to 0.04 w/v %, 0.002 to 0.04 w/v %, 0.00001 to 0.02 w/v %, 0.0001 to 0.02 w/v %, 0.0005 to 0.02 w/v %, 0.001 to 0.02 w/v %, 0.002 to 0.02 w/v %, 0.0007 to 0.02 w/v %, 0.0007 to 0.002 w/v %, 0.0007 to 0.005 w/v %, 0.001 to 0.005 w/v %, 0.00001 to 0.003 w/v %, or 0.0001 to 0.01 w/v %.

Isopropylmethylphenol has only been approved to be contained in, for example, cosmeceutical products (quasi-drugs), drugs as a bactericidal agent, in a concentration of 0.05 to 0.10 w/v % (PFSB/ELD Notification NO. 1225001, “List of active ingredients in so-called cosmeceutical products”), and cosmeceutical products and cosmetic products which comprise isopropylmethylphenol in a concentration of 0.00001 w/v % or more and 0.04 w/v % or less has hitherto been unknown.

The amount of the above-mentioned drugs and the like administered or fed may vary depending on the condition, weight, gender, age, or other factors of the subject. However, for oral administration or ingestion, the dose of isopropylmethylphenol is preferably 0.0001 mg/kg or more, and 100 mg/kg or less, more preferably 50 mg/kg or less, still more preferably 25 mg/kg or less, per adult per day. Furthermore, the dose is 0.0001 to 100 mg/kg, preferably 0.0001 to 50 mg/kg, still more preferably 0.0001 to 25 mg/kg.

Subjects to which the drugs and the like are administered or fed include a human in need of or desirous of the suppression of melanin hyperproduction on the skin, for example, a human desirous of the improvement in pigmentation, spots and freckles, and a human desirous of the suppression of the creation of pigmentation, spots and freckles after tanning, or desirous of skin-whitening.

With regard to the above-mentioned embodiment, the following embodiments of the present invention will be disclosed.

<1> A tyrosinase inhibitor comprising isopropylmethylphenol as an active ingredient.
<2> A melanin production inhibitor comprising isopropylmethylphenol as an active ingredient.
<3> A skin-whitening agent comprising isopropylmethylphenol as an active ingredient.
<4> A cosmetic product which comprises isopropylmethylphenol in a concentration of 0.00001 w/v % or more and 0.04 w/v % or less, 0.0001 w/v % or more and 0.04 w/v % or less, 0.0005 w/v % or more and 0.04 w/v % or less, 0.001 w/v % or more and 0.04 w/v % or less, 0.002 w/v % or more and 0.04 w/v % or less, 0.00001 w/v % or more and 0.02 w/v % or less, 0.0001 w/v % or more and 0.02 w/v % or less, 0.0005 w/v % or more and 0.02 w/v % or less, 0.001 w/v % or more and 0.02 w/v % or less, 0.002 w/v % or more and 0.02 w/v % or less, 0.0007 w/v % or more and 0.02 w/v % or less, 0.0007 w/v % or more and 0.002 w/v % or less, 0.0007 w/v % or more and 0.005 w/v % or less, 0.001 w/v % or more and 0.005 w/v % or less, 0.00001 w/v % or more and 0.003 w/v % or less, or 0.0001 w/v % or more and 0.01 w/v % or less.
<5> The cosmetic product of <4>, wherein the cosmetic product is a skin-whitening cosmetic.
<6> Use of isopropylmethylphenol for the production of a tyrosinase inhibitor.
<7> Use of isopropylmethylphenol for the production of a melanin production inhibitor.
<8> Use of isopropylmethylphenol for the production of a skin-whitening agent.
<9> Isopropylmethylphenol for use in tyrosinase inhibition.
<10> Isopropylmethylphenol for use in melanogenesis suppression.
<11> Isopropylmethylphenol for use in skin-whitening.
<12> A method for inhibiting tyrosinase comprising administering or feeding isopropylmethylphenol to a subject in need thereof.
<13> A method for suppressing melanogenesis comprising administering or feeding isopropylmethylphenol to a subject in need thereof.
<14> A method for skin-whitening comprising administering or feeding isopropylmethylphenol to a subject in need thereof.

### EXAMPLES

### Example 1

### Suppression of DOPA Oxidase Activity by isopropylmethylphenol in cell extract

### (1) Preparation of Cell Extract (Cell Lysate)

Normal human neonatal epidermal melanocytes (NHEMs; KURABO INDUSTRIES LTD.) were seeded in a T-175 flask, and was cultured at 37° C., in the presence of 5% CO₂. Medium 254 containing PMA-free growth supplement (HMGS) was used as a medium. The cells were harvested at 90% confluence, and then 10 mL of extraction buffer (0.1 M Tris-HCL (pH 7.2), 1% NP-40, 0.01% SDS, 100 μM PMSF, 1 μg/m aprotinin) was added to the cells. The cells were then treated with ultrasonication, and were centrifuged at 15000 r.p.m. for 10 minutes, and the supernatant was obtained.

### (2) Measurement of Tyrosinase Activity

Tyrosinase activity was measured using the cell extract mentioned above. DOPA oxidase activity was measured according to the following method, with reference to the MBTH method (Winder A. et al., 1991, Eur. J. Biochem. 198:317-326). 80 μL of Assay Buffer (4% dimethylformamide, 100 mM Sodium phosphate-buffered (pH 7.1)), 60 μL of 20.7 mM MBTH (3-methyl-2-benzothiazolinone hydrazone) solution, and 40 μL of 5 mM L-DOPA (L-dihydroxyphenylalanine) solution as a substrate, were added to each well of a 96-well plate. Then, 20 μL of the following test compounds were added to the wells so that the mixture reaches the final concentration shown in Table 1. 20 μL of the cell extract obtained from the step (1) was added to the well, and was allowed for reaction at 37° C. for 30 to 60 minutes. Then the color reaction was measured based on absorbance at 490 nm (N=3). The measurements are expressed as values relative to that of the control.

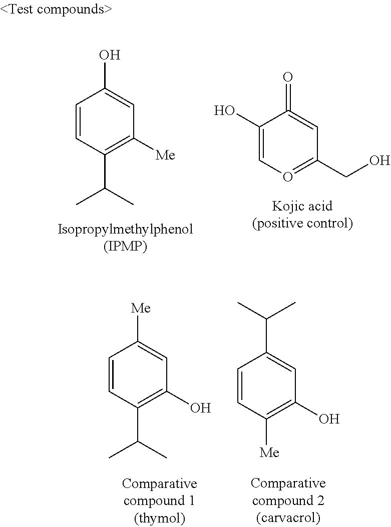

### (3) Results

The results are shown in Table 1.

Isopropylmethylphenol showed higher DOPA oxidase activity suppressive effect than kojic acid, depending on the concentration (0.00075 to 0.015 w/v %). Meanwhile, the comparative compound 1 (thymol) and the comparative compound 2 (carvacrol) did not show DOPA oxidase activity suppressive effect.

TABLE 1

| Evaluation | | IPMP | | |
|---|---|---|---|---|
| concentration | | (concentration | Comparative | Comparative |
| (μM) | Kojic acid | in w/v %) | compound 1 | compound 2 |
| 50 | 93.9 ± 1.5 | 83.1 ± 3.9 | 103.7 ± 0.9 | 102.1 ± 0.5 |
| | | (0.00075%) | | |
| 100 | 83.3 ± 1.6 | 62.9 ± 3.5 | 93.8 ± 1.6 | 96.9 ± 1.8 |
| | | (0.0015%) | | |
| 500 | 60.9 ± 1.9 | 32.8 ± 3.7 | 104.4 ± 0.8 | 99.6 ± 1.8 |
| | | (0.0075%) | | |
| 1000 | 42.2 ± 0.5 | 19.8 ± 1.2 | 105.4 ± 0.5 | 97.8 ± 1.9 |
| | | (0.015%) | | |

### Example 2

### Suppression of DOPA Oxidase Activity by Isopropylmethylphenol in Cultured Cells (1)

### (1) Cell Culture

Normal human neonatal epidermal melanocytes (NHEMs; KURABO INDUSTRIES LTD.) were seeded in a 96-well plate at a cell density of 1×10⁴ cells/well (100 μL/well), and was cultured at 37° C., in the presence of 5% CO₂. Medium 254 containing a PMA-free growth supplement (HMGS) was used as a medium.

After cultivating for 3 days, Endotheline-1 (ET-1), SCF, α-MSH, Histamine, and PGE₂, each prepared to be the final concentration of 1 nM in the medium, were added. The same test compounds used in Example 1 were also added so as to reach the final concentration. The mixture was cultured at 37° C. for 3 days, in the presence of 5% CO₂. The same amount of DMSO was added as a control.

### (2) Measurement of DOPA Oxidase Activity

After the completion of the cultivation, 20 L/well of alamar blue reagent (Invitrogen Corporation) was added. After incubating for 2 to 3 hours, the fluorescence intensity of the medium was measured to monitor the cell respiration activity. Then, the cells were washed with PBS, and 20 μL/well of extraction buffer (0.1 M Tris-HCL (pH 7.2), 1% NP-40, 0.01% SDS, 100 μM PMSF, 1 μg/m aprotinin) and 20 μL/well of Assay Buffer (4% dimethylformamide, 100 mM Sodium phosphate-buffered (pH 7.1)) were added. The cells were solubilized at 4° C. for 3 hours, and DOPA oxidase activity was measured. DOPA oxidase activity was measured according to the following method, with reference to the MBTH method (Winder A. et al., 1991, Eur. J. Biochem. 198:317-326).

80 μL of the above-mentioned Assay Buffer, 60 μL of 20.7 mM MBTH (3-methyl-2-benzothiazolinone hydrazone) solution, and 40 μL of 5 mM L-DOPA (L-dihydroxyphenylalanine) solution as a substrate, were added to each well of solubilized cell solution, and the mixture was allowed for reaction at 37° C. for 30 to 60 minutes. Then the color reaction was measured based on absorbance at 490 nm (N=3). The measurements are expressed as values relative to that of the control.

### (3) Results

The results are shown in Table 2.

Isopropylmethylphenol showed higher DOPA oxidase activity suppressive effect than kojic acid, depending on the concentration. Meanwhile, the comparative compound 1 (thymol) and the comparative compound 2 (carvacrol) did not show DOPA oxidase activity suppressive effect. From the monitoring of the cell respiration activity by the alamar blue method, it was confirmed that the concentration of isopropylmethylphenol which showed DOPA oxidase activity suppressive effect did not affect cell growth.

TABLE 2

| | | DOPA oxidase | | Alamar blue | |
|---|---|---|---|---|---|
| Test compounds | | activity | | (cell toxicity) | |
| (concentration (μM)) | | AVE | SD | AVE | SD |
| Kojic acid | 10 | 102.5 | 12.3 | 118.3 | 2.3 |
| | 100 | 94.7 | 14.7 | 114.4 | 4.2 |
| IPMP | 10 (0.00015 w/v %) | 74.8 | 4 | 94.1 | 4.9 |
| | 100 (0.0015 w/v %) | 50.6 | 4.4 | 127 | 3.7 |
| Comparative | 10 | 98.6 | 1.5 | 84.9 | 2.5 |
| compound 1 | 100 | 95 | 9.3 | 104.4 | 7.3 |
| Comparative | 10 | 99.3 | 4.3 | 94.2 | 12.2 |
| compound 2 | 100 | 96.3 | 8.4 | 115.2 | 7.7 |

### Example 3

### Suppression of DOPA Oxidase Activity by Isopropylmethylphenol in Cultured Cells (2)

Isopropylmethylphenol was added so as to reach the final concentration shown in Table 3, and the DOPA oxidase activity was measured in the same manner as in Example 2. The results are shown in Table 3 altogether.

TABLE 3

| | | DOPA oxidase | | Alamar blue | |
|---|---|---|---|---|---|
| Test compounds | | activity | | (cell toxicity) | |
| (concentration (μM)) | | AVE | SD | AVE | SD |
| IPMP | 0.67 (0.00001 w/v %) | 73.7 | 9.5 | 84.9 | 0.5 |
| | 6.7 (6.0001 w/v %) | 56.5 | 4.7 | 85.4 | 4.6 |
| | 67 (0.001 w/v %) | 33.9 | 2.6 | 78.9 | 5.0 |
| | 167.5 (0.0025 w/v %) | 25.6 | 0.9 | 77.0 | 3.8 |

From table 3, it was recognized that isopropylmethylphenol shows DOPA oxidase activity suppressive effect depending on the concentration, within the concentration range of 0.00001 to 0.0025 w/v %. It was also confirmed that, within the concentration range, the suppressing effect did not affect cell growth.

1. A tyrosinase inhibitor comprising isopropylmethylphenol as an active ingredient.

2. A melanin production inhibitor comprising isopropylmethylphenol as an active ingredient.

3. A skin-whitening agent comprising isopropylmethylphenol as an active ingredient.

4. A cosmetic product comprising isopropylmethylphenol in a concentration of 0.00001 w/v % or more and 0.04 w/v % or less.

5. A cosmetic product comprising isopropylmethylphenol in a concentration of 0.00001 w/v % or more and 0.02 w/v % or less.

6. A cosmetic product comprising isopropylmethylphenol in a concentration of 0.00001 w/v % or more and 0.01 w/v % or less.

7. A cosmetic product comprising isopropylmethylphenol in a concentration of 0.00001 w/v % or more and 0.001 w/v % or less.

8. The cosmetic product according to claim 4, wherein the cosmetic product is a skin-whitening cosmetic.

9. Use of isopropylmethylphenol for the production of a tyrosinase inhibitor.

10. Use of isopropylmethylphenol for the production of a melanin production inhibitor.

11. Use of isopropylmethylphenol for the production of a skin-whitening agent.

12. A method for inhibiting tyrosinase comprising administering or feeding isopropylmethylphenol to a subject in need thereof.

13. A method for suppressing melanogenesis comprising administering or feeding isopropylmethylphenol to a subject in need thereof.

14. A method for skin-whitening comprising administering or feeding isopropylmethylphenol to a subject in need thereof.
